(19) United States
(12) Patent Application Publication
(10) Pub. No.: US 2008/0249375 A1
(43) Pub. Date: Oct. 9, 2008

(54) Arrangement and Method for Evaluating Operational Effectiveness of Implantable Medical Electrode Leads for Different Lead Placements

(76) Inventor: Martin Obel, Danderyd (SE)

Correspondence Address: SCHIFF HARDIN, LLP;PATENT DEPARTMENT, 6600 SEARS TOWER, CHICAGO, IL, 60606-6473, US

(21) Appl. No.: 11/666,964

(22) PCT Filed: Nov. 2, 2004
(86) PCT No.: PCT/SE04/01601

§ 371 Date: Mar. 31, 2008

(51) Int. Cl. A61B 5/02

(52) U.S. Cl. 600/301; 600/547; 600/508

(57) ABSTRACT

In a method and an arrangement for evaluating operational effectiveness of an implantable medical device for different lead placements associated with the medical device, a measuring unit records signals that are characteristic of cardiac activity at respectively different lead positions, and these signals are stored. A processor accesses the stored signals and, from the stored signals, determines a measure of cardiac activity at each of the lead positions. The recorded signals may be intracardiac ECG signals, surface ECG signals, heart sound signals obtained from a microphone, or impedance signals. The lead position at which the best hemodynamic behavior of the heart is identified from the analysis of the stored signals, and is determined as being the optimum site for placement of the electrode leads.

### BACKGROUND OF THE INVENTION

1. Field of the Invention

The present invention generally relates to implantable medical devices such as implantable cardiac pacemakers and in particular to a device and a method for obtaining information related to the heart pumping activity at different lead positions within a heart and for using the information to evaluate the different lead positions in order to identify the optimal position with respect to the heart pumping activity.

2. Description of the Prior Art

The technology of cardiac pacemakers has developed in sophistication and functionality over the years. In general, cardiac pacemakers are designed to control the heart by correcting or compensating for various heart abnormalities which can be encountered in human patients. For example, cardiac pacemakers may provide therapeutic stimulation to the heart by delivering therapeutic pulses such as pacing, cardioversion or defibrillation pulses.

Commonly, the pulses are delivered to the heart via electrodes disposed on implantable leads coupled to the pacemaker. The pacemaker performs various sensing and pulsing functions by receiving and delivering signals through the leads. The placements of the electrodes, i.e. the leads, with respect to one or more cardiac locations are such so that the desired electrical functions such as stimulation or heart signal sensing are ensured. For example, the leads may position the electrodes with respect to one or more cardiac locations so that the pacemaker can deliver pulses to the appropriate locations of the heart.

Leads may be placed in one or more of a variety of different cardiac locations. In particular, the placement of the leads may be dependent on the cardiac conditions of the patient and the therapy to be delivered.

A proper placement of the leads, i.e. the electrodes, is essential because both the desired electrical functions, i.e. stimulation or heart signal sensing, and the desired heart muscle reaction (activity propagation) are dependent on the position of the lead. However, it is often difficult to determine whether a lead has been properly positioned and adequate tissue contact has been achieved. Today, pacemaker leads are normally checked during the implantation to ensure satisfactory electrical performance by use of a PSA (pacemaker system analyzer). A PSA is an external equipment connected to the implanted leads. In general three electrode and lead properties are checked by use of a PSA to be satisfactory:
1. Stimulation threshold. That the required electrical stimulation energy to activate the heart is low enough.
2. Heart signal. That the spontaneous heart signal picked up by the lead system is of enough amplitude.
3. Lead impedance. That the conductive path of the lead(s) together with body fluids and tissue is in physical good condition.

Furthermore, it is often difficult to get the lead to the proper or desired site, specially the left ventricular lead through the coronary sinus, partly due to technical difficulties and anatomic differences, but also due to the lack of a standard procedure for identifying the optimal or best stimulation spot for that specific patient. One method for left ventricular lead placement includes so called venogram, where a fluoro-visible dye is injected into the veins so that the veins are visible using a fluoroscopic device.

Thus, there is a need of an improved device and method that, in an efficient way, is capable of identifying the optimal position of the lead or the leads within a heart with respect to the heart pumping activity during an implantation procedure of an implantable medical device.

### SUMMARY OF THE INVENTION

An object of the present invention is to provide a simplified and improved device and method for finding the optimal lead placement for one or more leads with respect to heart pumping activity during an implantation procedure. After the lead or leads have been positioned at an optimal site they are disconnected from the device and connected to the permanent pulse generator to be implanted. The device may be an improved PSA or a separate device.

The above object is achieved in accordance with the invention by a device for evaluating positions of one or several medical leads during an implantation procedure, the medical lead comprising at least one electrode for stimulating and sensing. The device has a measuring arrangement that records signals with the lead to be implanted at different positions, a storage unit that stores the signals, and a processing unit that determines the lead position resulting in the most favorable hemodynamics of the heart based on a measure of the recorded signals at each position. The recorded signals may be one or several of the following:
Impedance signals measured from the lead or leads under implantation at different lead positions.
Heart sound signals measured from a microphone placed on a patient's body with the lead or leads at different positions.
Surface ECG signals with the lead or leads to be implanted at different positions.
Intracardiac signals measured from the lead or leads under implantation at different lead positions.

The above object also is achieved in accordance with the invention by a method for evaluating positions of one or several medical leads during an implantation procedure. The method includes the steps of: recording signals at different lead positions; storing the signals; and determining the lead position resulting in the most favorable hemodynamics of heart based on a measure of the signals at each position.

The above object also is achieved in accordance with the present invention by a computer program product in the form of a computer-readable medium encoded with a data structure that, when loaded into a computer or a processor of a device as described above, causes the device to operate in the manner described above, to implement the inventive method.

The invention is based on the use of signals characteristic of the heart pumping activity to evaluate different positions of a medical lead or medical leads during an implantation procedure in order to identify the optimal site or placement for the lead or the leads with respect to a desired heart muscle reaction. That is, physiologic parameters reflecting hemodynamic performance are derived and evaluated for each lead position in order to determine the optimal site.

This solution provides several advantages over existing solutions. One advantage is that the optimal site for placing the lead (or the leads) can be accurately determined with respect to anatomic differences of a specific patient, the specific therapy to be delivered and the desired heart muscle reaction (activity propagation).

Another advantage is that the lead site optimization is incorporated into the implant procedure. This provides for significant time savings in comparison with prior art solutions, which, in turn, reduces the risk for infection because the time required for the implantation procedure is decreased.

In a preferred embodiment of the present invention, the bipolar right ventricular impedance is measured in order to detect the contraction of the right ventricle. At the same time the left ventricular impedance is measured in order to detect the contraction of the left ventricle. The left ventricular impedance may be measured between a left ventricular electrode placed in a coronary vein and a right ventricular electrode. The impedance changes in this configuration are mainly due to the variable left ventricular volume during the heart cycle. The lead position or positions resulting in the best synchrony between the right ventricular contraction and the left ventricular contraction is considered as the best lead position. It should be noted, however, that the V-V interval should be optimized before any tests are performed to evaluate the merits of the actual lead position is evaluated. For biventricular pacing the left ventricular lead position is generally the most important lead position. In a typical case the RV lead is placed in an apical position while the LV lead is placed at different positions in the coronary vein system to find the position giving the best hemodynamic performance. If applicable, the AV-interval between an atrial event and the following ventricular stimulations should also be optimized before the tests to determine the merits of a lead position.

According to a further embodiment the contractibility of the heart muscle is measured and the lead position or lead positions giving the highest value of the contractility is selected as the best lead position or positions. This embodiment utilizes the fact that rate of change of the impedance during contraction provides a measure of contractility. At least one of the LV or RV impedance should be measured to determine the contractility.

According to a further embodiment the ejection fraction of the heart muscle is measured and the lead position or lead positions giving the highest value of the ejection fraction is selected as the best lead position or positions. This embodiment utilizes the fact that the difference between minimum and maximum value of the impedance provides a measure of the ejection fraction.

According to still another embodiment an interval preceding the ventricular ejection is measured and the lead position or positions giving the shortest interval is deemed to be the best lead position or positions. A segment of the impedance waveform starting with a paced QRS complex or a ventricular stimulation and terminating at a predetermined impedance value is recorded. The duration of this interval has a proportionality with the Pre-ejection time Period (PEP). Details of how the this measurement is performed and how it relates to PEP is provided in U.S. Pat. No. 4,773,401. The lead position or positions providing the shortest Pre-ejection interval is deemed to be the best lead position.

According to a further embodiment of the present invention, the cardiac output is determined at each lead position and the lead position resulting in the highest cardiac output, which is the volume of blood in liters ejected by the heart per minute, is determined as the lead position resulting in the most favorable hemodynamics of the heart. Cardiac output is determined by the product of stroke volume and heart rate. It is well known that a measure of stoke volume can be determined through impedance measurements.

According to a further embodiment the surface ECG is recorded with the leads to be implanted at different positions. The surface ECG provides information on the synchrony between the right and left ventricles. If the lead position or lead positions are not optimal the paced QRS will have a longer duration than normal even if V-V interval between the stimulating pulses for the two ventricles is optimized. The shortest possible QRS can be achieved with the V-V interval optimized and with the two leads in an optimal position. The lead position giving the shortest duration of the paced QRS should be searched. The morphology of the paced QRS could also be analyzed to determine the optimal lead position.

The surface ECG may be achieved from chest leads or from extremity leads.

According to still another embodiment a heart sound microphone can be placed on the patient's chest. This would allow a measurement of the patient's heart sound at different lead positions. This arrangement would allow to identify the lead position or lead positions giving the shortest Pre-Ejection period time (PEP). The lead position giving the shortest PEP would be the most optimal lead position or lead positions hemodynamically.

According to another embodiment of the present invention, the lead position resulting in the lowest value of the quotient between PEP and left ventricular ejection time (LVET) is determined to be the lead position resulting in the most favorable hemodynamics of the heart. LVET is affected by the contractility of the myocardium and by outflow obstructions at the left ventricle. At a degraded contractility with a low stroke volume LVET will decrease, whilst it will be lengthened at outflow obstructions, such as aortic stenosis, and at a large central stroke volume. PEP tends to increase at, inter alia, cardiac insufficiency. As with PEP, LVET must be corrected with respect to the heart rate. The PEP/LVET quotient reflects the function of the left ventricle in a more efficient way than the individual components and is not dependent on the heart rate. Hence, the quotient PEP/LVET provides an efficient and reliable measure of the heart pumping activity.

Preferably, the AV interval between stimulation of the atrium and the ventricle and/or the VV interval between stimulation of the right and left ventricles are optimized before the measurement related to the determination of the performance of the actual lead position is performed.

### BRIEF DESCRIPTION OF THE DRAWINGS

FIG. 1 shows a device in use according to the present invention for evaluating a lead position during an implantation procedure.

FIG. 2 is block diagram of a device for evaluating the position of a medical lead under implantation through impedance measurements according to the present invention.

FIG. 3 is block diagram of a device for evaluating the position of a medical lead under implantation through ECG measurements according to the present invention.

FIG. 4 is block diagram of a device for evaluating the position of a medical lead under implantation through heart sound measurements according to the present invention.

FIG. 5 shows the morphology of a surface QRS under normal conditions and with Right Bundle Branch Block (RBBB) and with Left Bundle Branch Block (LBBB).

FIG. 6 is a flow chart describing the principle steps of the process of evaluating different position of a lead of an implantable medical device to find an optimal lead site in accordance with the present invention.

### DESCRIPTION OF THE PREFERRED EMBODIMENTS

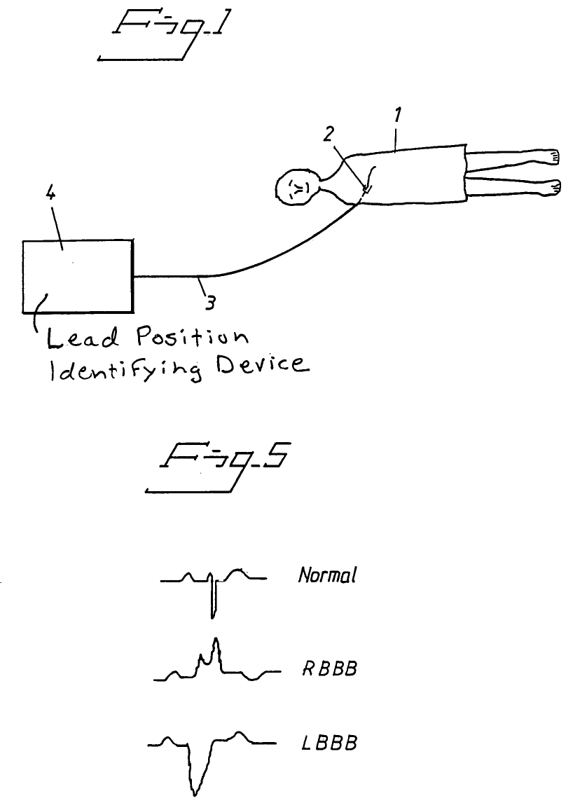

FIG. 1 illustrates the situation during the implantation during a pacemaker implantation. The patient 1 is placed on a table during the implantation procedure. The lead or leads 2 to be implanted are inserted via a suitable vein. Via this vein the leads are placed in the patient's heart under fluoroscopic supervision. Possible positions for different leads could be right atrium, the right ventricle, the left atrium or the left ventricle via the coronary sinus. Via the coronary sinus a lead can be placed in a suitable coronary vein for pacing and sensing the left ventricle of the heart. The position of the lead for pacing and sensing the left ventricle is very important for the hemodynamics. The device 4 is generally known as a Pacing System Analyzer (PSA). The device 4 is connected to the lead to be implanted via an adaptor cable 3. The device 4 is according to current practice used to measure certain important data related to the lead to be implanted. This data may be R-wave or P-wave amplitude or stimulation threshold. Further the lead impedance is frequently measured. According to the present invention the device 4 may be used to collect data on the hemodynamic performance of each lead position. The lead or leads are located at different positions and the data related to the hemodynamic performance are collected for each lead position.

FIG. 2 shows a preferred embodiment of the present invention which utilizes sampled impedance measurements to determine the hemodynamic performance of a lead position. The pulse generating unit 6 delivers pacing pulses to one or several leads under implantation. The leads may be located in one or several of the right atrium, the left atrium, the right ventricle or the left ventricle. When the leads have been placed in a first position the impedance measuring unit 5 is activated to sample impedance values from one or several of the implanted leads. The sampling of the lead impedances is controlled by a microprocessor 8. The measured impedances vary with the blood volumes and the mechanical movements of the heart. In order to be able to follow the changes of volume or contraction pattern of a heart chamber the sampling frequency of the impedance sampling unit 5 should be 64 samples/s or more. The sampled values are stored in a memory 7. When the desired number of lead positions have been tested and the samples have been stored in the memory 7, the samples can be analyzed through execution of an a analyzing software to determine which lead position that gives the most favorable hemodynamics. The result of the analysis may be provided to a user through the presentation unit 9.

Several different algorithms can be used to analyze the hemodynamic qualities of a given lead position. It is generally accepted that the contractility is a good measure of hemodynamics and that a high contractility is an indicator of good hemodynamic performance. In one embodiment the lead position giving the highest rate of change of the impedance is selected as the most favorable lead position. U.S. Pat. No. 5,800,467 discloses that with greater contractibility of the heart the greater volume of blood is pumped by the heart for any given heart rate. It is also disclosed that by measuring an impedance within a ventricle an indication of the contractility can be provided. The impedance can be measured as a bipolar impedance within a ventricle, preferably the right ventricle. If the impedance is measured between an epicardially located electrode on the left ventricle and an endocardially located electrode in the right ventricle an indication of left ventricular volume and left ventricular contractility can be achieved.

The algorithm to find the most favorable electrode position may also be based on detection of ejection fraction. The lead position resulting in the highest value of Ejection Fraction (EF) would be considered as the most favorable lead position. Ejection fraction can be determined by dividing stroke volume with the end-diastolic volume. U.S. Pat. No. 5,514,171 discloses how ejection fraction can be determined based on impedance measurements.

Another algorithm to find a favorable electrode position would be to search a position giving the shortest pre-ejection interval which is a well-known indicator hemodynamic performance. U.S. Pat. No. 4,773,401 discloses that the interval between a paced QRS until the right ventricular impedance crosses a zero axis in a positive direction is good measure of a pre-ejection time interval. The lead position giving the shortest pre-ejection interval is a favorable lead position.

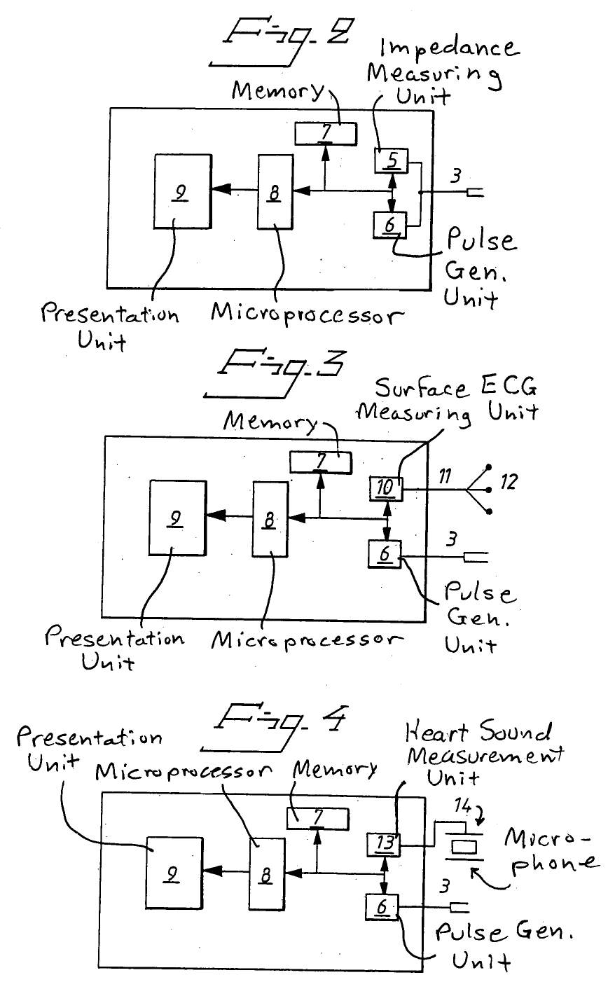

FIG. 3 shows a preferred embodiment of the present invention which utilizes sampled surface ECG measurements to determine the hemodynamic performance of a lead position. The pulse generating unit 6 delivers pacing pulses to one or several leads under implantation. The leads may be located in one or several of the right atrium, the left atrium, the right ventricle or the left ventricle. When the leads have been placed in a first position the surface ECG measuring unit 10 is activated to sample a surface ECG. The surface ECG electrodes are placed on the patient's body or on his arms and legs. From the surface electrogram one can determine if the atria and the ventricles are depolarized in an appropriate sequence through an analysis of a sequence of the electrogram. The width or duration of a paced QRS is a well-known indication of if the synchronization between the right and left ventricles is appropriate. After the sampled ECG has been stored in the memory 7 the microprocessor can measure the width of a paced QRS. The lead position or lead positions giving the shortest duration of the paced QRS is considered as the most favorable lead position.

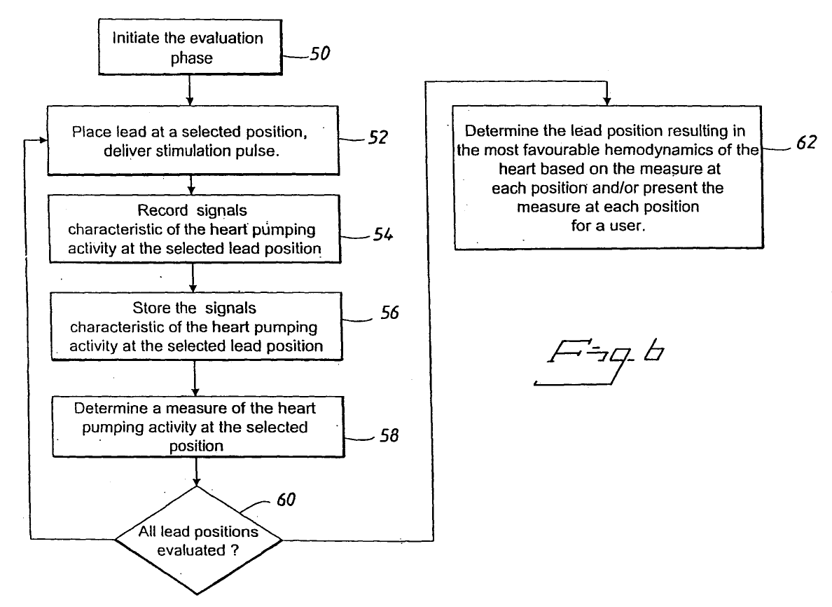

In a further embodiment the morphology of the paced QRS is analyzed and the lead position which yields a surface ECG indicating the most synchronized contraction is considered as the most favorable lead position. In FIG. 5 is indicated how the surface ECG morphology differs between a normal QRS and a QRS morphology indicating Right Bundle Branch Block (RBBB) and Left Bundle Branch Block (LBBB) respectively. The most favorable lead position is indicated by the QRS which from a morphology standpoint is closest to a normal QRS. The sampled surface ECGs are stored in the memory 7. The microprocessor executes software to analyze the morphology of the stimulated QRS with different lead positions to identify the most favorable lead position. The QRS morphology analysis may be through a comparison with predetermined criteria or through a comparison with one or several templates.

FIG. 4 shows a further embodiment in which heart sounds are measured and sampled through a heart sound microphone 13 placed on a patient's body. Through well-known analysis of heart sounds caused by the opening and closing of the aortic valve can be detected. The Pre-Ejection Period defined as the time interval between delivery of a stimulation pulse or detection of a QRS until the opening of the aortic valve is a well-known criteria of hemodynamic performance. The pulse generating unit 6 comprises means for detecting QRS/P-waves and for emitting stimulation pulses to the atria and to the ventricle of a patient. The heart sounds at different lead positions are measured and sampled through microphone 14, heart sound measurement unit 13 and stored in memory 7. Through an analysis of stored heart sounds by execution of a suitable software in microprocessor 8 the lead position giving the shortest PEP is identified as the most favorable lead position.

In a further improved embodiment the Left Ventricular Ejection Time is identified as the time interval between the opening and closing of the aortic valve. The lead position giving the lowest value of the quotient PEP/LVET is considered as a favorable lead position. The value of the quotient at different lead positions is determined through execution of a suitable software by microprocessor 8. The quotient PEP/LVET is a well-known indicator of the function of the left ventricle. The normal value of the quotient PEP/LVET is 0.30-0.40 while it in situations with poor hemodynamics can be as high as 0.60.

FIG. 5 shows the morphology of a surface ECG at a normal condition and at Right Bundle Branch Block (RBBB) and at Left Bundle Branch Block (LBBB). With a proper lead position and a proper stimulation sequence QRS shape will be more similar to a normal QRS. If the stimulated QRS is sampled and stored in memory 7 for several different lead positions this information can obviously be used to select the lead position giving the most favorable conditions. Morphological criteria as well as other criteria such as QRS time duration can be used.

With reference now to FIG. 6, a flow chart of the principles of the process of evaluating different positions of one or more leads according to the present invention during implantation of the medical device will be described. First, at step 50, a test phase is activated by the user. This can be performed by activating an evaluation or optimization sequence or program stored in the memory 7 of the evaluation device. As discussed above, the position or the placement of the lead is essential for the functions of the medical device as well as regards to obtaining the desired heart muscle reaction and finding or identifying the optimal lead position with respect to the heart activity is often difficult. Therefore, a number of different lead positions may have to be tested and evaluated during the implantation procedure of the medical device in order to find a placement of the lead that gives the optimal heart activity at stimulation.

Then, at step 52, the physician places the lead or leads 2 at a first position by means of a guide wire or other suitable technique, one or several stimulation pulses are delivered at the selected position using the pulse generator 6. Thereafter, at step 54, signals characteristic of the heart activity are recorded. For example, it may be intracardiac impedance signals indicating the left ventricular and/or right ventricular volume. That is, physiologic parameters reflecting hemodynamic performance are derived for each lead position. In addition, IEGM signals and/or surface ECG signals may be recorded. Also heart sounds may be recorded via a microphone 13 placed on the patient's body. Heart sounds are particularly useful to determine the opening and closing of heart valves. There are a number of different parameters that can be used as hemodynamical indicators of the heart pumping activity including the pre-ejection period (PEP), the quotient between PEP and left ventricular ejection time (LVET), the coordination between the contraction of the left ventricle and the contraction of the right ventricle, the ventricular contractility (measured as rate of change of impedance), or the cardiac output. Subsequently, at step 56, the signals characteristic of the heart activity at the selected lead position are stored in the memory 7 of the evaluation device. As will be discussed below, the signal characteristic of the heart activity may depend on which parameter that is used in the evaluation procedure. Preferably, the AV interval between stimulation of the atrium and the ventricle and/or the VV interval between stimulation of the right and left ventricles are optimized before the measurement related to the determination of the performance of the actual lead position is performed.

At step 58, the processing means determines a measure or a score value of the heart activity for the actual lead position using the recorded signal data. The measure or score value may be presented for the user on the presentation unit 9. At step 60, the user may select whether another lead position is to be evaluated or tested. If yes, the above mentioned steps 52-58 are repeated. If no, the processing means determines which lead position that results in the most favorable hemodynamics of the heart based on the determined or calculated measure for each lead position at step 62. A number of different parameters can be used for this determination. For example, the lead position resulting in the shortest pre-ejection time period (PEP) may be determined to be the optimal site with respect to the hemodynamics of the heart. The measure at each lead position together with the lead position resulting in the most favorable hemodynamics of the heart can be presented for the user visually on the presentation unit 9.

Any of the embodiments disclosed above may be used to determine the optimal lead position.

Preferably, the AV interval between stimulation of the atrium and the ventricle and/or the VV interval between stimulation of the right and left ventricles are optimized before the measurement related to the determination of the performance of the actual lead position is performed in the above mentioned different embodiments of the present invention employing different approaches to derive physiologic parameters reflecting hemodynamic performance for different lead positions in order to determine the optimal lead site.

Although an exemplary embodiment of the present invention has been shown and described, it will be apparent to those of ordinary skill in the art that a number of changes, modifications, or alterations to the inventions as described herein may be made. Thus, it is to be understood that the above description of the invention and the accompanying drawings is to be regarded as a non-limiting example thereof and that the scope of protection is defined by the appended patent claims. As an example, many of the functions described above may be obtained and carried out by suitable software comprise in a microchip, an ASIC, or the like data carrier.

I claim as my invention:

1-41. (canceled)

42. In a device comprising a plurality of electrode leads configured to interact in vivo with tissue of the patient, during said electrode leads being respectively, selectively positionable at a plurality of different positions in the patient during an implantation procedure, the improvement of an arrangement for evaluating effectiveness of operation of said electrode leads with said electrode leads placed at respectively different positions in the patient, said arrangement comprising:
a measuring unit that obtains samples, with said electrode leads being respectively placed at different ones of said positions, selected from the group consisting of impedance samples, microphonic heart sound samples, surface ECG samples, and intracardiac ECG samples;
a storage unit connected to said measuring unit that stores said samples; and
a processor having access to said storage unit that analyzes said samples and, from said samples, identifies one of said positions of said electrode leads that produces most favorable hemodynamic behavior of the heart of the patient.

43. A device as claimed in claim 42 wherein said measuring unit obtains said impedance samples, and wherein said processor analyzes said impedance samples and determines a rate of change in impedance represented by said samples and, from said rate of change of impedance, identifies, as said one of said positions, a position of said electrode leads at which a highest ventricular contractibility occurs.

44. A device as claimed in claim 42 wherein said measuring unit obtains said impedance samples, and wherein said processor identifies a maximum and a minimum of impedance represented by said impedance samples, and identifies said one of said positions as a position of said electrode leads at which a highest ventricular ejection fraction, determined from said maximum and said minimum of said impedance, occurs.

45. A device as claimed in claim 42 wherein said measuring unit obtains said impedance samples, and wherein said processor identifies, as said one of said positions, a position of said electrode leads at which a most synchronized contraction of right and left chambers of the heart occurs, determined from said impedance.

46. A device as claimed in claim 42 comprising a stimulation pulse generator connected to said electrode leads that emits stimulation pulses that are delivered to said tissue via said electrode leads to pace the heart of the patient, and wherein said measuring unit obtains said impedance samples, and wherein said processor determines an impedance of the heart from said impedance samples and determines a pre-ejection period from said impedance as a time interval starting with a paced QRS complex or a ventricular stimulation and ending when impedance in the ventricle reaches a predetermined value, and identifies said one of said lead positions as a position of said electrode leads at which a minimum of said pre-ejection period occurs.

47. A device as claimed in claim 42 wherein said measuring unit obtains said impedance samples, and wherein said processor determines impedance of the heart from said impedance samples and determines stroke volume of the heart from said impedance, and identifies said one of said positions as a position of said electrode leads at which a highest stroke volume occurs.

48. A device as claimed in claim 42 comprising a stimulation pulse generator connected to said electrode leads that emits stimulation pulses that are delivered to the heart of the patient via said electrode leads, and wherein said measuring unit obtains said surface ECG, and wherein said processor executes a morphology analysis of said surface ECG and, from said morphology analysis, identifies said one of said positions as a position of said electrode leads at which a stimulated QRS complex of the heart has a morphology closest to a reference QRS complex.

49. A device as claimed in claim 42 comprising a stimulation pulse generator connected to said electrode leads that emits stimulation pulses that are delivered to the heart of the patient via said electrode leads, and wherein said measuring unit obtains said surface ECG samples, and wherein said processor analyzes said surface ECG samples to determine a paced QRS time duration therefrom, and identifies said one of said positions as a position of said electrode leads at which a shortest QRS time duration occurs.

50. A device as claimed in claim 42 comprising a stimulation pulse generator connected to said electrode leads that emits stimulation pulses that are delivered to the heart of the patient via said electrode leads, and wherein said measuring unit obtains said heart sound samples and said intracardiac ECG samples and wherein said processor, from said heart sound samples and said intracardiac ECG samples, determines a pre-ejection period duration as a time interval from a paced or sensed ventricular event until opening of the aortic valve, and identifies said one of said positions as a position of said electrode leads at which a shortest pre-ejection period duration occurs.

51. A device as claimed in claim 50 wherein said processor further determines left ventricular ejection time from said heart sound samples and said intracardiac ECG samples, and determines the quotient of said pre-ejection time duration and said left ventricular ejection time, and identifies said one of said positions as a position of said electrode leads at which said quotient has a lowest value.

52. A device as claimed in claim 42 comprising a stimulation pulse generator connected to said electrode leads that emits stimulation pulses that are delivered to the heart of the patient via said electrode leads, and wherein said measuring unit obtains said heart sound samples and said surface ECG samples and wherein said processor, from said heart sound samples and said surface ECG samples, determines a pre-ejection period duration as a time interval from a paced or sensed ventricular event until opening of the aortic valve, and identifies said one of said positions as a position of said electrode leads at which a shortest pre-ejection period duration occurs.

53. A device as claimed in claim 52 wherein said processor further determines left ventricular ejection time from said heart sound samples and said surface ECG samples, and determines the quotient of said pre-ejection time duration and said left ventricular ejection time, and identifies said one of said positions as a position of said electrode leads at which said quotient has a lowest value.

54. A device as claimed in claim 42 wherein one of said electrode leads is configured for positioning in the right ventricle of the heart of the patient.

55. A device as claimed in claim 54 wherein another of said electrode leads is configured for positioning at a location selected from the group consisting of the left ventricle, in the coronary sinus, and a coronary vein.

56. A device as claimed in claim 42 comprising a stimulation pulse generator connected to said electrode leads that emits stimulation pulses, and wherein a first of said electrode leads is configured for placement in the right ventricle of the heart of the patient and a second of said electrode leads is configured for placement in the left ventricle of the heart of the patient, said first and second of said electrode leads respectively delivering stimulation pulses from said stimulation pulse generator to stimulate the right ventricle and the left ventricle.

57. A device as claimed in claim 56 comprising a control unit that operates said stimulation pulse generator and is connected to said processor, said control unit optimizing the VV interval between stimulation of the right ventricle and stimulation of the left ventricle before enabling identification of said one of said positions by said processor.

58. A device as claimed in claim 42 comprising a stimulation pulse generator connected to said electrode leads that emits stimulation pulses, and wherein a first of said electrode leads is configured for placement in an atrium of the heart of the patient and a second of said electrode leads is configured for placement in a ventricle of the heart of the patient, said first and second of said electrode leads respectively delivering stimulation pulses from said stimulation pulse generator to stimulate the atrium and the ventricle.

59. A device as claimed in claim 58 comprising a control unit that operates said stimulation pulse generator and is connected to said processor, said control unit optimizing the AV interval between stimulation of the ventricle and stimulation of the atrium before enabling identification of said one of said positions by said processor.

60. A device as claimed in claim 42 comprising a stimulation pulse generator connected to said electrode leads that emits stimulation pulses, and wherein a first of said electrode leads is configured for placement in the right ventricle of the heart of the patient, and a second of said electrode leads is configured for placement in the left ventricle of the heart, and a third of said electrode leads is configured for placement in an atrium of the heart, and wherein said stimulation pulse generator emits stimulation pulses that are respectively delivered to the right ventricle, the left ventricle and the atrium via said first, second and third of said electrode leads to stimulate the right ventricle, the left ventricle and the atrium.

61. A device as claimed in claim 59 comprising a control unit connected to said stimulation pulse generator and to said processor, said control unit optimizing an AV interval between stimulation of the atrium and one of said ventricles, and a VV interval between stimulation of the right ventricle and the left ventricle, before enabling identification of said one of said positions by said processor.

62. A method for operating a device comprising a plurality of electrode leads configured to interact in vivo with tissue of the patient, said electrode leads being respectively, selectively positionable at a plurality of different positions in the patient during an implantation procedure for the device, said method evaluating effectiveness of said operation of said electrode leads with said electrode leads placed at respectively different positions in the patient, by steps comprising:
obtaining samples, with said electrode leads being respectively placed at different ones of said positions, selected from the group consisting of impedance samples, microphonic heart sound samples, surface ECG samples, and intracardiac ECG samples;
storing said samples; and
accessing the stored samples and automatically electronically analyzing said samples to, from said samples, identify one of said positions of said electrode leads that produces most favorable hemodynamic behavior of the heart of the patient.

63. A method as claimed in claim 62 comprising obtaining said impedance samples, and analyzing said impedance samples to determine a rate of change in impedance represented by said samples and, from said rate of change of impedance, identifying, as said one of said positions, a position of said electrode leads at which a highest ventricular contractibility occurs.

64. A method as claimed in claim 62 comprising obtaining said impedance samples, and identifying a maximum and a minimum of impedance represented by said impedance samples, and identifying said one of said positions as a position of said electrode leads at which a highest ventricular ejection fraction, determined from said maximum and said minimum of said impedance, occurs.

65. A method as claimed in claim 62 comprising obtaining said impedance samples, and identifying, as said one of said positions, a position of said electrode leads at which a most synchronized contraction of right and left chambers of the heart occurs, determined from said impedance.

66. A method as claimed in claim 62 wherein said device comprises a stimulation pulse generator connected to said electrode leads that emits stimulation pulses that are delivered to said tissue via said electrode leads to pace the heart of the patient, and wherein said method comprises obtaining said impedance samples, and determining an impedance of the heart from said impedance samples and determining a pre-ejection period from said impedance as a time interval starting with a paced QRS complex or a ventricular stimulation and ending when impedance in the ventricle reaches a predetermined value, and identifying said one of said lead positions as a position of said electrode leads at which a minimum of said pre-ejection period occurs.

67. A method as claimed in claim 62 comprising obtaining said impedance samples, and determining impedance of the heart from said impedance samples and determining stroke volume of the heart from said impedance, and identifying said one of said positions as a position of said electrode leads at which a highest stroke volume occurs.

68. A method as claimed in claim 62 wherein said device comprises a stimulation pulse generator connected to said electrode leads that emits stimulation pulses that are delivered to the heart of the patient via said electrode leads, and wherein said method comprises obtaining said surface ECG, and automatically electronically executing a morphology analysis of said surface ECG and, from said morphology analysis, identifying said one of said positions as a position of said electrode leads at which a stimulated QRS complex of the heart has a morphology closest to a reference QRS complex.

69. A method as claimed in claim 62 wherein said device comprises a stimulation pulse generator connected to said electrode leads that emits stimulation pulses that are delivered to the heart of the patient via said electrode leads, and wherein said method comprises obtaining said surface ECG samples, automatically electronically analyzing said surface ECG samples to determine a paced QRS time duration therefrom, and identifying said one of said positions as a position of said electrode leads at which a shortest QRS time duration occurs.

70. A method as claimed in claim 62 wherein said device comprises a stimulation pulse generator connected to said electrode leads that emits stimulation pulses that are delivered to the heart of the patient via said electrode leads, and wherein said method comprises obtaining said heart sound samples and said intracardiac ECG samples and from said heart sound samples and said intracardiac ECG samples, determining a pre-ejection period duration as a time interval from a paced or sensed ventricular event until opening of the aortic valve, and identifying said one of said positions as a position of said electrode leads at which a shortest pre-ejection period duration occurs.

71. A method as claimed in claim 70 comprising further determining left ventricular ejection time from said heart sound samples and said intracardiac ECG samples, and determining the quotient of said pre-ejection time duration and said left ventricular ejection time, and identifying said one of said positions as a position of said electrode leads at which said quotient has a lowest value.

72. A method as claimed in claim 62 wherein said device comprises a stimulation pulse generator connected to said electrode leads that emits stimulation pulses that are delivered to the heart of the patient via said electrode leads, and wherein said method comprising obtaining said heart sound samples and said surface ECG samples and from said heart sound samples and said surface ECG samples, determining a pre-ejection period duration as a time interval from a paced or sensed ventricular event until opening of the aortic valve, and identifying said one of said positions as a position of said electrode leads at which a shortest pre-ejection period duration occurs.

73. A method as claimed in claim 72 comprising further determining left ventricular ejection time from said heart sound samples and said surface ECG samples, and determining the quotient of said pre-ejection time duration and said left ventricular ejection time, and identifying said one of said positions as a position of said electrode leads at which said quotient has a lowest value.

74. A method as claimed in claim 62 comprising positioning one of said electrode leads in the right ventricle of the heart of the patient.

75. A method as claimed in claim 74 comprising positioning another of said electrode leads at a location selected from the group consisting of the left ventricle, in the coronary sinus, and a coronary vein.

76. A method as claimed in claim 62 wherein said device comprises a stimulation pulse generator connected to said electrode leads that emits stimulation pulses, and wherein said method comprising placing a first of said electrode leads in the right ventricle of the heart of the patient and a second of said electrode leads in the left ventricle of the heart of the patient and, with said first and second of said electrode leads respectively delivering stimulation pulses from said stimulation pulse generator to stimulate the right ventricle and the left ventricle.

77. A method as claimed in claim 76 comprising automatically electronically optimizing the VV interval between stimulation of the right ventricle and stimulation of the left ventricle before identifying said one of said positions.

78. A method as claimed in claim 62 wherein said device comprises a stimulation pulse generator connected to said electrode leads that emits stimulation pulses, and wherein said method comprises placing a first of said electrode leads in an atrium of the heart of the patient and placing a second of said electrode leads in a ventricle of the heart of the patient, and from said first and second of said electrode leads, respectively delivering stimulation pulses from said stimulation pulse generator to stimulate the right ventricle and the left ventricle.

79. A method as claimed in claim 76 comprising automatically electronically optimizing the AV interval between stimulation of the ventricle and stimulation of the atrium before identifying said one of said positions.

80. A method as claimed in claim 62 wherein said device comprises a stimulation pulse generator connected to said electrode leads that emits stimulation pulses, and wherein said method comprises placing a first of said electrode leads in the right ventricle of the heart of the patient, and placing a second of said electrode leads is configured in the left ventricle of the heart, and placing a third of said electrode leads in an atrium of the heart, and, from said stimulation pulse generator, emitting stimulation pulses that are respectively delivered to the right ventricle, the left ventricle and the atrium via said first, second and third of said electrode leads to stimulate the right ventricle, the left ventricle and the atrium.

81. A method as claimed in claim 79 comprising automatically electronically optimizing an AV interval between stimulation of the atrium and one of said ventricles, and a VV interval between stimulation of the right ventricle and the left ventricle, before identifying said one of said positions.

82. A computer-readable medium encoded with a data structure for use with a device comprising a plurality of electrode leads configured to interact in vivo with tissue of the patient, said electrode leads being respectively, selectively positionable at a plurality of different positions in the patient during an implantation procedure, a measuring unit, a storage unit, and a processor, said data structure, when said medium is loaded into said processor, causing said processor to evaluate effectiveness of operation of said electrode leads with said electrode leads placed at respectively different positions in the patient, by:
operating said measuring unit to obtain samples, with said electrode leads being respectively placed at different ones of said positions, selected from the group consisting of impedance samples, microphonic heart sound samples, surface ECG samples, and intracardiac ECG samples;
storing said samples in said storage; and
accessing said storage unit and analyzing said samples and, from said samples, identifying one of said positions of said electrode leads that produces most favorable hemodynamic behavior of the heart of the patient.
